(19) United States
(12) Patent Application Publication
(10) Pub. No.: US 2010/0274083 A1
(43) Pub. Date: Oct. 28, 2010

(54) HUMAN BODY COMMUNICATION SYSTEM AND METHOD

(75) Inventors: Chang Hee Hyoung, Daejeon (KR); Sung Weon Kang, Daejeon (KR); Jung Hwan Hwang, Daejeon (KR); Sung Eun Kim, Seoul (KR); Jin Kyung Kim, Daejeon (KR); Tae Wook Kang, Daejeon (KR); Kyung Soo Kim, Daejeon (KR); Jung Bum Kim, Daejeon (KR); Hyung II Park, Daejeon (KR); In Gi Lim, Daejeon (KR); Ki Hyuk Park, Daejeon (KR); Jae Hoon Shim, Daejeon (KR)

Correspondence Address: AMPACC Law Group, 3500 188th Street S.W., Suite 103, Lynnwood, WA, 98037, US

(73) Assignee: ELECTRONICS AND TELECOMMUNICATIONS RESEARCH INSTITUTE, DAEJEON (KR)

(21) Appl. No.: 12/808,178

(22) PCT Filed: Sep. 19, 2008
(86) PCT No.: PCT/KR08/05576

§ 371 Date: Jun. 14, 2010

(30) Foreign Application Priority Data
Dec. 17, 2007 (KR) 10-2007-0132324

(51) Int. Cl. A61B 1/04; H04B 7/00

(52) U.S. Cl. 600/109; 455/41.2; 375/130; 375/E01.001

(57) ABSTRACT

There is provided a human body communication system including: a wireless capsule injected into the body to acquire image information of the interior of the body and transmitting a signal with respect to the acquired information to the outside of the body from the inside of the body by using an electrode; and a portable data recorder performing, from the outside of the body, RF communication with the wireless capsule by the medium of the body, receiving the signal transmitted from the wireless capsule through an electrode in contact with the human body, processing the received signal into RF data, and storing the RF data.

### TECHNICAL FIELD

The present invention relates to a human body communication system and method, and more particularly, to a human body communication system and method that transmits information acquired inside the body by using a wireless capsule inserted into the body to a recording device outside the body by using the body as a transmission medium.

This work was supported by the IT R&D program of MIC/IITA[2006-S-072-02, Controller SoC for Human Body Communications].

### BACKGROUND ART

Recently, an apparatus capable of reducing pain in subjects and automatically performing endoscopy and treatment of the subjects has attracted attention. To respond to the attention, a system in which an automatic vision system and a RF data transmission system are integrated into a small tablet has been developed. This system has been subject to medical assessment and been approved by the United States (US). Expensive products to which the system is applied have already been released and used.

One of the techniques according to the related art is U.S. Pat. No. 5,604,531 that discloses a swallowable capsule type wireless endoscope system. The capsule-type wireless endoscope system includes an image capturing apparatus (imaging apparatus), a transmitter, an illumination apparatus, and a power source having a battery. The image capturing apparatus includes a micro camera inside a capsule. The micro camera keeps transmitting clear images of the stomach and intestines to an image recording device fixed to the waist of a subject by using the transmitter. Then, the recording device transmits data, which are recorded and then stored, to a hospital. A doctor in the hospital reads the image data by using a computer and analyzes the read image data. In this way, the doctor can check conditions of the stomach and intestines. With the use of the capsule type endoscope, the subject does not need to stay in the hospital, pain in the subject is relieved, and the entire digestive system can be checked by taking the capsule only once.

In general, information acquisition is periodically performed. A bidirectional communication between a communication device in the capsule and an external apparatus allows more accurate data acquisition. Therefore, functions related to this are being added. Further, the related art promotes a forward movement and a backward movement that are caused by contraction using an electric propulsion apparatus or electrostimulation that allows the capsule type endoscope to stay at a predetermined position for the accurate examination or move in an inverse direction as well as a movement caused by rhythmic, called peristalsis in the digestive system.

However, in the techniques according to the related art, the information acquired inside the body can be transmitted through the communication between the communication device inside the capsule and the external apparatus. However, the information acquired inside the body includes high-resolution images. High power is consumed to transmit the high-resolution images to the external apparatus. Since the techniques according to the related art use limited power inside the capsule, it may be difficult to transmit the high-resolution images without any problem.

Further, as shown in FIG. 1, a capsule type endoscope 10 using an existing human body communication uses at least two electrodes 11 having a potential difference. Therefore, in order to transmit information acquired inside the body to an apparatus 20 outside the body, the at least two electrodes 11 need to be stably in contact with the human body. To this end, FIG. 2 shows examples of various electrode arrangements of the existing capsule type endoscope that needs two electrodes having a potential difference in order to stably maintain contacts as examples of electrode arrangements.

However, since the capsule type endoscope has no autonomous power supply, it is difficult to stably maintain at least two electrodes. Further, when the existing capsule type endoscope uses an existing RF communication device for the communication with the external apparatus, a carrier frequency needs to be high, and thus peripheral circuits of A/D converters require high power consumption.

### DISCLOSURE OF INVENTION

### Technical Problem

An aspect of the present invention provides a human body communication system and method that reduces power consumption and obtains high-speed communication by using human body communication to acquire good-quality information for a long period of time instead of an existing RF method used to perform communication between a wireless capsule inside the body and a recording apparatus positioned outside the body.

### Technical Solution

According to an aspect of the present invention, there is provided a human body communication system including: a wireless capsule injected into the body to acquire image information of the interior of the body and transmitting a signal with respect to the acquired information to the outside of the body from the inside of the body by using an electrode; and a portable data recorder performing, from the outside of the body, human body communication with the wireless capsule by the medium of the body, receiving the signal transmitted from the wireless capsule through an electrode in contact with the human body, processing the received signal into data, and storing the data.

The wireless capsule may include an information acquiring unit acquiring the image information; a human body communication unit modulating the signal with respect to the acquired information; an electrode formed at the surface of the wireless capsule and applying the modulated signal to the body to transmit the modulated signal to the outside; and a battery supplying power.

The portable data recorder may include an electrode in contact with the surface of the body and receiving the signal transmitted through the body; a human body communication unit demodulating the signal received through the electrode; and a data processing unit recording data with respect to the demodulated signal in a storage unit.

According to another aspect of the present invention, there is provided a human body communication method between a wireless capsule and a portable recording apparatus in a human body communication system having the wireless capsule injected into the body and the portable recording apparatus formed outside the body, the method including: acquiring image information detected inside the body by the wireless capsule; modulating a signal with respect to the acquired information by the wireless capsule; applying the modulated signal to the body through an electrode in contact with the body by the wireless capsule; receiving the signal applied to the body by the medium of the body by the portable recording apparatus; and demodulating the received signal and recording the demodulated signal in a storage unit by the portable recording apparatus.

### Advantageous Effects

Therefore, according to the exemplary embodiment of the present invention, a human body communication system and method can reduce power consumption by removing circuits required for modulation and demodulation by using a selective spread spectrum method, but not by using a frequency modulation method, obtain a high transmission rate by directly transmitting a broadband digital signal (baseband signal) to the human body, and stably ensure desired data by two-way communication.

Further, since electrodes having the same phases are formed in the wireless capsule injected into the body, contacts can be more stably maintained as compared when a plurality of electrodes are formed, and the contacts can be stably maintained through various arrangements of the electrodes having the same phases.

### BRIEF DESCRIPTION OF THE DRAWINGS

FIG. 1 is a diagram illustrating communication between a capsule type endoscope according to the related art and an external apparatus;

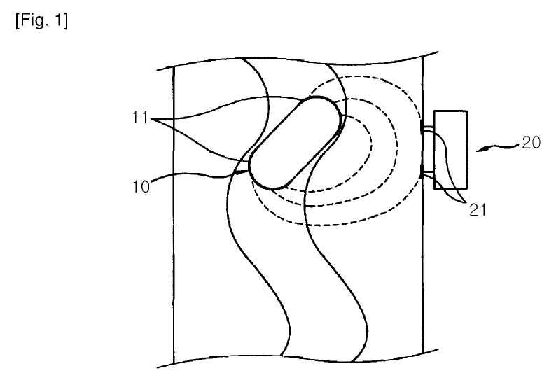

FIG. 2 is a diagram illustrating electrode arrangements of the capsule type endoscope according to the related art;

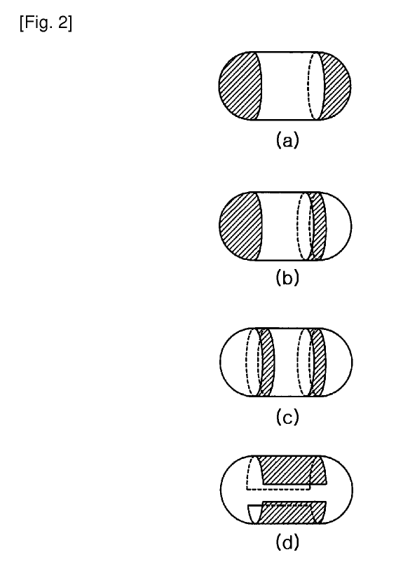

FIG. 3 is a diagram illustrating a configuration of a human body communication system using human body communication according to an exemplary embodiment of the present invention;

FIG. 4 is a diagram illustrating electrode arrangements of a wireless capsule according to an exemplary embodiment of the present invention; and

FIG. 5 is a diagram illustrating different electrode arrangements of the wireless capsule according to the exemplary embodiment of the present invention;

### BEST MODE FOR CARRYING OUT THE INVENTION

Hereinafter, exemplary embodiments of the present invention will now be described in detail with reference to the accompanying drawings. However, in description of operation principles associated with the embodiments of the present invention, detailed description of a known art or configuration is omitted because it may obscure the spirit of the present invention unnecessarily.

A human body communication system according to exemplary embodiments of the invention can be applied to medical equipment, for example, a capsule type endoscope that is injected into the human body. The configuration of the human body communication system according to the exemplary embodiment of the present invention will now be described in detail with reference to the accompanying drawings.

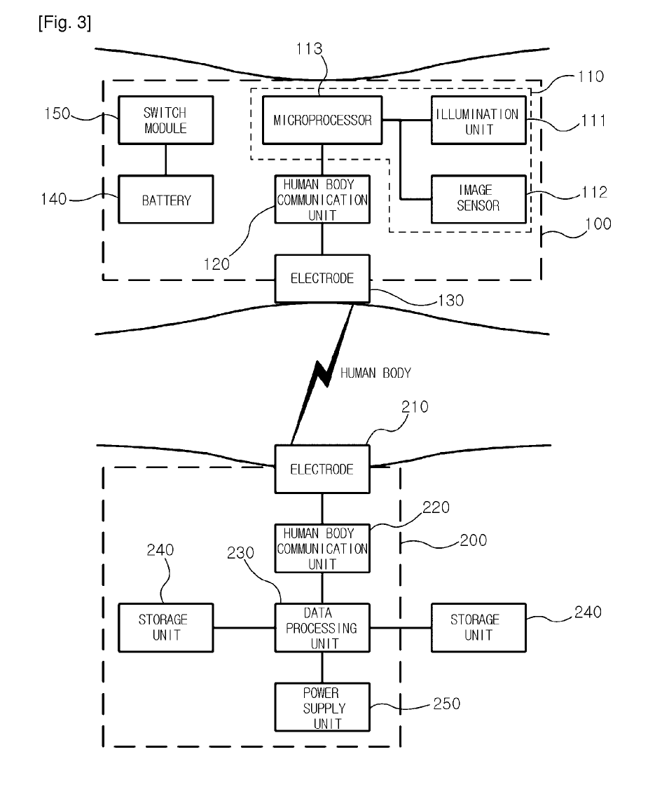

FIG. 3 is a diagram illustrating a configuration of a human body communication system according to an exemplary embodiment of the present invention.

Referring to FIG. 3, the human body communication system may include a wireless capsule 100 and a portable data recorder 200. The wireless capsule 100 is injected into the human body. The portable data recorder 200 is located outside the human body and records data. The wireless capsule 100 and the portable data recorder 200 perform two-way communication to receive and transmit data by the medium of the human body. In order to minimize the power consumption, the wireless capsule has only the transmitter unit of the human body communication and the portable data recorder 200 has only the receiver unit of human body communication.

The wireless capsule 100 may include an information acquiring unit 110, a human body communication unit 120, an electrode 130, a battery 140, and a switch module 150.

The information acquiring unit 110 includes an illumination unit 111, an image sensor 112, and a microprocessor 113. The illumination unit 111 has a light source. The image sensor 112 acquires image information by using the light source. The microprocessor 113 processes the acquired image information.

The human body communication unit 120 modulates a signal with respect to the acquired information transmitted from the microprocessor 113 and applies the modulated signal to the human body through the electrode 130. The human body communication unit 120 can stably perform communication under electromagnetic interference from various peripheral electronic devices. Further, in order to obtain a higher data transmission rate with the limited battery 140, the human body communication unit 120 modulates the signal by using a selective spread spectrum method, but not by using a continuous frequency modulation method. Therefore, the human body communication unit 120 can communicate with the portable data recorder through the human body without performing RF modulation of a broadband digital signal (baseband signal).

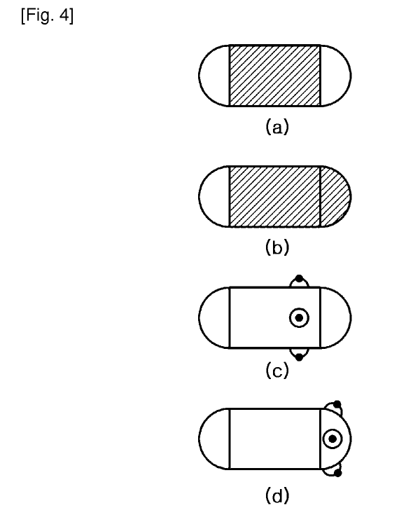

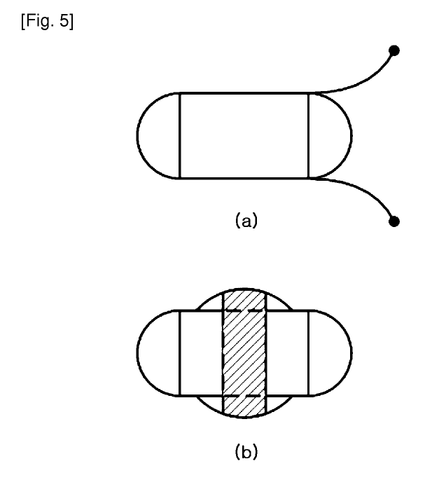

The electrode 130 is formed at the surface of the wireless capsule 100. The electrode 130 is in contact with the human body and applies the signal to the human body. In this embodiment of the invention, the description is made of a case in which there is one electrode 130. However, in order to stably maintain contacts inside the body, there may be a plurality of electrodes that have the same phases. FIG. 4 shows examples of the one or more electrodes formed on the wireless capsule 100. FIGS. 4A and 4B show examples of the arrangement of one electrode. FIGS. 4C and 4D show examples of the arrangement of a plurality of electrodes having the same phases. Further, the shape of the electrode can be varied as shown in FIG. 5.

The portable data recorder 200 includes an electrode 210, a human body communication unit 220, a data processing unit 230, a storage unit 240, and a power supply unit 250. The electrode 210 is formed at the surface of the human body and receives the signal applied to the human body. The human body communication unit 220 demodulates the signal received through the electrode 210. The data processing unit 230 records data with respect to the demodulated signal. The storage unit 240 is formed at the inside or the outside.

The human body communication system having the above-described configuration can stably ensure information acquired inside the body at the high transmission rate through communication between the wireless capsule inside the body and the external portable data recorder outside the body. A human body communication method in the above-described human body communication system will now be described in detail.

The wireless capsule 100 is injected into the human body. When the switch module 150 operates and is in an ON state, power is supplied through the battery 140 to operate the wireless capsule 100.

When the power is supplied, the light source of the illumination unit 111 generates the light source. The image sensor 112 acquires necessary image information inside the body by using the light source and transmits the acquired image information to the microprocessor 113. Then, the microprocessor 113 processes the transmitted image information and transmits the processed image information to the human body communication unit 120.

The human body communication unit 120 modulates the transmitted image information by using a selective spread spectrum method and then, transmits the modulated signal to the electrode 130 so that the signal is applied to the electrode 130 in contact with the human body. The modulated signal output through the electrode 130 is transmitted from the stomach and intestines to the portable data recorder 200 positioned outside the human body by the medium of the human body.

Then, the portable data recorder 200 receives the signal through the electrode 210 in contact with the human body. At this time, the received signal is weak and transmitted to the human body communication unit 220. Then, the human body communication unit 220 demodulates the received weak signal and transmits the demodulated signal to the data processing unit 230.

The data processing unit 230 records the demodulated signal to the storage unit 240 that is positioned at the inside or outside.

While the present invention has been shown and described in connection with the exemplary embodiments, it will be apparent to those skilled in the art that modifications and variations can be made without departing from the spirit and scope of the invention as defined by the appended claims.

1. A human body communication system comprising:
a wireless capsule injected into the body to acquire image information of the interior of the body and transmitting a signal with respect to the acquired information to the outside of the body from the inside of the body by using an electrode; and
a portable data recorder performing, from the outside of the body, communication with the wireless capsule through the body as a medium, receiving the signal transmitted from the wireless capsule through an electrode in contact with the human body, processing the received signal into data, and storing the data.

2. The human body communication system of claim 1, wherein the wireless capsule comprises:
an information acquiring unit acquiring the image information;
a human body communication unit modulating the signal with respect to the acquired information;
an electrode formed at the surface of the wireless capsule and applying the modulated signal to the body to transmit the modulated signal to the outside; and
a battery supplying power.

3. The human body communication system of claim 2, wherein the information acquiring unit comprises an illumination unit having a light source, an image sensor acquiring the image information, and a microprocessor processing the acquired information.

4. The human body communication system of claim 2, wherein the human body communication unit modulates the signal with respect to the acquired information by using a selective spread spectrum method.

5. The human body communication system of claim 2, wherein when the electrode comprises at least two electrodes, the at least two electrodes have the same phases.

6. The human body communication system of claim 1, wherein the portable data recorder comprises:
an electrode in contact with the surface of the body and receiving the signal transmitted through the body;
a human body communication unit demodulating the signal received through the electrode; and
a data processing unit recording data with respect to the modulated signal in a storage unit.

7. A human body communication method between a wireless capsule and a portable recording apparatus in a human body communication system having the wireless capsule injected into the body and the portable recording apparatus formed outside the body, the method comprising:
acquiring image information detected inside the body by the wireless capsule;
modulating a signal with respect to the acquired information by the wireless capsule;
applying the modulated signal to the body through an electrode in contact with the body by the wireless capsule;
receiving the signal applied to the body by the medium of the body by the portable recording apparatus;
demodulating the received signal; and
recording the demodulated signal in storage unit by the portable recording apparatus.

8. The human body communication system of claim 7, wherein the modulating a signal with respect to the acquired information by the wireless capsule comprises modulating the signal with respect to the acquired information by using a selective spread spectrum method.
